(19) United States
(12) Patent Application Publication
(10) Pub. No.: US 2019/0295683 A1
(43) Pub. Date: Sep. 26, 2019

(54) SYSTEMS AND METHODS FOR NON-HORMONAL FEMALE CONTRACEPTIVE DRUG TARGET IDENTIFICATION AND PRIORITIZATION

(71) Applicant: Celmatix Inc., New York, NY (US)

(72) Inventors: Piraye Yurttas Beim, New York, NY (US); David Emlyn Parfitt, New York, NY (US); Mark Adams, Norwalk, CT (US)

(21) Appl. No.: 16/365,339

(22) Filed: Mar. 26, 2019

Related U.S. Application Data
(60) Provisional application No. 62/648,309, filed on Mar. 26, 2018.

(51) Int. Cl. G16B 5/00; C12Q 1/68

(52) U.S. Cl. CPC G16B 5/00; C12Q 1/68

(57) ABSTRACT

The present disclosure is directed to systems and methods for identifying and prioritizing targets, specifically key genetic loci underlying one or more fertility disorders, for non-hormonal female contraceptive drug development.

### CROSS-REFERENCE TO RELATED APPLICATION

This application claims priority to and the benefit of U.S. Provisional Patent Application Ser. No. 62/648,309, filed Mar. 26, 2018, the contents of which are incorporated by reference herein in its entirety.

### FIELD

The invention generally relates to systems and methods for identifying and prioritizing targets, specifically key genetic loci underlying one or more fertility disorders, for non-hormonal female contraceptive drug development.

### BACKGROUND

Birth control, also known as contraception and fertility control, includes methods or devices used to prevent pregnancy. The most effective means of birth control are considered to be sterilization (i.e., tubal ligation in females), as well as intrauterine devices (IUDs) or other implantable devices. The next most effective means of birth control includes hormone-based methods, including, but not limited to, oral pills, patches, injections, and intravaginal rings. Oral contraceptives, commonly referred to as birth control pills, either contain a combination of estrogen and a progestin or contain progestogen-only, both formulations prevent fertilization mainly by inhibiting ovulation and thickening cervical mucous.

While considered to be an effective means of preventing pregnancy and further provide additional benefits, the use of hormonal contraceptives may also result in unwanted, serious, and sometimes life threatening side effects, based, at least in part, on the fact that virtually every organ system in the female body is affected by, and responsive to, the hormone compounds in contraceptive formulations. For example, common negative side effects may include intermenstrual spotting, nausea, breast tenderness, headaches and migraine, weight gain, mood changes, decreased libido, intermittent vaginal discharge. Furthermore, most birth control pills increase a female's chance of developing a blood clot by about three to four times as a result of the hormones increasing the levels of clotting factors. Such risks increase with age and length of exposure to these the oral contraceptives. For women who live in communities that are not supportive of contraception to begin with, the fear of serious side effects can further discourage women from utilizing these drugs. In the absence of alternatives, many women still seek surgical sterilization procedures, which are permanent and carry their own risk of complications. Furthermore, women in the developing world who do develop serious complications from contraceptive drug use may lack access to the care needed to detect and treat such life-threatening events.

### SUMMARY

The present disclosure is directed to systems and methods for identifying and prioritizing targets for non-hormonal female contraceptive drug development. The non-hormonal female contraceptive drug may be a small molecule, antibody, recombinant protein, or other formulation. The drug may impact any biological process or processes, including those regulating hormonal function.

In particular, the present disclosure includes a system configured to perform genome-wide association studies of all known human genes that have the potential to be pre-fertilization drug targets for female contraceptive development. The systems and methods of the invention identify genes that possess a genetic or functional association with any phenotype and/or condition related to female fertility and/or reproductive health. The systems and methods of the invention identify drugs that may target at least one of those biological process or events occurring during the reproductive cycle, for example at any point from the formation of the human oocyte to the implantation of the embryo into the uterus.

In particular, the system carries out a genome-wide, comprehensive evaluation and screening to uncover genetic factors related to one or more fertility disorders, such as endometriosis, polycystic ovary syndrome, and premature ovarian failure (also known as primary ovarian insufficiency (POI)), for example. The genetic factors identified by the system and methods of the invention may regulate any biological process related to oocyte development and maturation, fertilization, or implantation of an early embryo into a uterus. These biological processes include, but are not limited to folliculogenesis, oogenesis, oocyte maturation, and ovulation of an egg capable of being fertilized, as well as fertilization, luteinization, endometrial proliferation, and any process by which the endometrium becomes receptive to an embryo.

Fertility-related health conditions can occur from changes to any number of these biological processes, and may have genetic causes. Hence, the systems and methods of the invention also involve identifying genetic factors associated with one or more fertility-related conditions in women, such as those involving ovulatory dysfunction, for example, premature ovarian insufficiency (POI), polycystic ovary syndrome, and early menopause, as well as those involving changes to endometrial function, for example endometriosis.

The evaluation and screening processes account for multiple data layers including, but not limited to, temporal and spatial gene expression patterns, pathway-analysis, and protein function. Genomic loci, or biological molecules, such as proteins, metabolites, microRNAs, lncRNAs identified by methods of the invention are annotated with data from a plurality of data streams. Data streams can include any type of evidence that associates a putative genetic target with a relevant phenotype or condition. Target-phenotype association data streams include, but are not limited to genetic association of a target with condition, gene expression data indicating that the target is dysregulated during a pathological state, animal models where the target is modified, and pathway- or systems-level biological frameworks describing the target in disease or normal states. Data streams may also include any type of evidence that helps predict the druggability and/or tractability of the target, as well as the potential side effect profile if the target is drugged. Target profile data streams include, but are not limited to RNA/protein tissue specificity, and interpreted data from UniProt, HPA, PDBe, DrugEBllity, ChEMBL, Pfam, InterPro, Complex Portal, DrugBank, Gene Ontology, and BioModels.

The system is configured to rank the identified targets using quantification and the strength of evidence relating them to a relevant biological process or condition. For example, each independent target-phenotype data element within a given data stream may be assigned a score between 0-1 indicating the strength of the association. Each data stream may also be assigned a score between 0-1 that is a result of a harmonic sum function including all data elements within a data stream. An overall association score may be calculated, for example the score may be calculated using a harmonic sum function to add all target-phenotype data stream scores. If requirements for targets change, the systems and methods of the invention modify the weighting used to rank the targets to attain a desired output.

Methods of the invention include combining component variables to calculate the evidence association score for each target-phenotype combination. These variables may include relative occurrence of evidence supporting a particular target-disease combination (e.g., how often the target is observed to altered in case versus control subjects), the predicted functional consequence or severity of the effect of altering the target (e.g., as observed in animal models of the target, or as indicated by the odds ratio or relative risk of the alteration in the target and the phenotype or trait), or the overall confidence in the observations that constitute the target disease evidence (e.g., as indicated by the p-value of individual observations).

Upon identifying genetic factors, specifically key genetic loci underlying one or more fertility disorders, the system is configured to correlate the identified genetic factors with known metabolic pathways and signaling networks, particularly those pathways and networks likely to contain one or more druggable targets (i.e., a biological target, such as a protein, that is known to or is predicted to bind with high affinity to a drug). The system further identifies, from a database of known drug/gene interactions, one or more therapeutic candidates from existing pharmacopeia. The potential targets can then be validated via pre-clinical laboratory partners and/or in silico informatics and analysis techniques. Accordingly, a pathological basis of infertility is identified (i.e., one or more targets, including genetic factors and/or a pathway thought to be altered in a female fertility disorder) and used as a basis for developing a non-hormonal contraceptive drug in a female who otherwise has normal fertility (i.e., no known fertility disorders). More specifically, based on the identified and prioritized targets, a non-hormonal contraceptive drug can be developed and designed in so as to modulate an otherwise normal functioning signal pathway associated with a menstrual cycle, for example, to thereby alter the cycle and ultimately act as a contraceptive.

In one aspect, the present disclosure includes a method for screening for a non-hormonal female contraceptive therapeutic. The method includes identifying a gene or pathway known to function in female reproductive biology, exposing the gene or pathway to a candidate drug, and selecting, as a clinical candidate for a non-hormonal female contraceptive, a candidate non-hormonal female contraceptive drug that modulates the gene or pathway. The gene or pathway is thought to be altered in the context of any female ovulatory phenotype or trait. The selected candidate non-hormonal female contraceptive drug may upregulate or downregulate a protein associated with the gene or pathway. The process of identifying the gene or pathway comprises evaluating and screening at least one of temporal and spatial gene expression patterns, pathway analysis, and protein function.

In other aspects, the method includes identifying a gene or pathway thought to be altered in a female fertility disorder, exposing the gene or pathway to a candidate drug, and selecting, as a clinical candidate for a non-hormonal female contraceptive, a candidate non-hormonal female contraceptive drug that modulates the gene or pathway. The selected candidate non-hormonal female contraceptive drug may upregulate or downregulate a protein associated with the gene or pathway. The female fertility disorder is selected from the group consisting of endometriosis, polycystic ovary syndrome, and premature ovarian failure or primary ovarian insufficiency (POI). The process of identifying the gene or pathway comprises evaluating and screening at least one of temporal and spatial gene expression patterns, pathway analysis, and protein function.

In some embodiments, the method includes identifying a plurality of genes or pathways known to function in female reproductive biology. In other aspects, the method may include identifying a plurality of genes or pathways thought to be altered in the context of any female ovulatory phenotype or trait. In yet other aspects, the method includes identifying a plurality of genes or pathways thought to be altered in a female fertility disorder. The method may further include prioritizing the identified plurality of genes based on a predictive correlation with contraceptive efficacy. The step of prioritizing the identified plurality of genes may include ranking each of the identified plurality of genes based, at least in part, on attributes of each of the identified plurality of genes considered to be associated with contraceptive efficacy, wherein a higher ranking may correspond to a more positive correlation with contraceptive efficacy. The specific attributes may include, for example, genes known to be disrupted in females experiencing infertility that is refractory to in vitro fertilization treatment.

The identified gene may be associated with at least one of ADA, AGT, AKT1, ALDOA, AMBP, AMD1, ANXAS, APC, APOA1, APOE, AR, AREG, ATM, ATR, BAX, BCL2, BCL2L1, BDNF, BMP3, BMP4, BMP6, BMP7, BRCA1, BRCA2, BSG, CASP1, CBS, CCLS, CCND1, CCND2, CD19, CD28, CDKN2A, CGBS, COMT, CP, CRHR1, CSF1, CSF2, CX3CL1, CXCR4, CYP11A1, CYP19A1, CYP1A1, DDIT3, DHFR, DNMT1, DPYD, EGR1, ESR1, ESR2, FANCG, FASLG, FDXR, FGFR1, GALT, GATA4, GCK, GGT1, GNRH1, GRN, GSTA1, HBA2, HMOX1, HSD3B2, HSF1, ICAM1, IGF1, IGF1R, IGFBP3, IGFBP4, IL10, IL13, IL1B, ILS, IL6, IL8, IRF1, ITGAV, KIT, KITLG, LEP, LIF, LIFR, MAPK1, MAPK3, MAPK8, MAPK9, MDK, MDM2, MITF, MLH1, MSH2, MST1, MTHFR, MVP, MX1, MYC, NAT1, NCAM1, NOS3, NR5A1, NTRK1, NTRK2, PARP1, PCNA, PGK1, PGR, PRKCB, PRLR, PTGS1, PTGS2, QDPR, SELL, SLC28A1, STATS, STAT6, S, LT1E1, TBXA2R, TG, TNF, TOP2A, TP53, TPMT, TSHB, TYMS, VDR, VEGFA, XDH genes. In other embodiments, the identified gene may be one or more of the genes listed in Table 1.

Accordingly, the systems and methods of the present disclosure allow for the development of new contraceptive drugs (i.e., non-hormonal contraceptives) with minimal side effects and a more precise mechanism of action, as compared with current hormone-based contraceptives. Furthermore, the present invention has the potential to have a broader impact on women's health beyond contraception, as contraceptives are often used for non-contraceptive purposes in women with reproductive conditions such as endometriosis and polycystic ovary syndrome. The elucidation of the genetic basis of female fecundity and fertility disorders, particularly the discovery of the key genetic loci underlying these disorders, holds great promise for the identification of novel targets for drug development and therapeutics. More specifically, a better understanding of the crucial molecular pathways underlying human fecundity and fertility guides the next generation of targeted, non-hormonal contraceptives.

### BRIEF DESCRIPTION OF THE DRAWINGS

Features and advantages of the claimed subject matter will be apparent from the following detailed description of embodiments consistent therewith, which description should be considered with reference to the accompanying drawings.

FIG. 1 illustrates a computer system for implementing methodologies of the present disclosure.

FIG. 2 illustrates various stages of data processing consistent with the systems and methods of the present disclosure, particularly for carrying out the genome-wide, comprehensive evaluation and screening to uncover genetic factors related to one or more fertility disorders, including the determination of specific genes and associated metabolic pathways or signaling networks, and prioritization of such genes based on further processing and refinement.

FIG. 3 is an enlarged view of various stages of a reproductive cycle, namely the menstrual cycle, to which various genes and metabolic pathways or signaling networks are related.

FIG. 4 is an enlarged view of a network of genetic factors, non-genetic factors and biological processes directly or indirectly associated with ovulation.

FIGS. 5A-5J are enlarged views of the connections between genetic factors, non-genetic factors and biological processes.

FIGS. 6A and 6B are enlarged views of the gene prioritization tables used in illustrating the ranking of any given identified gene in view of additional processing of related data.

FIG. 7 illustrates an exemplary pathway of the fertility disorder known as primary ovarian insufficiency (POI).

FIG. 8 illustrates an example of a data stream of IVF-related phenotypes and outcomes associated with different genetic alterations for use in identifying candidate targets for non-hormonal contraceptives that affect ovarian or endometrial function.

For a thorough understanding of the present disclosure, reference should be made to the following detailed description, including the appended claims, in connection with the above-described drawings. Although the present disclosure is described in connection with exemplary embodiments, the disclosure is not intended to be limited to the specific forms set forth herein. It is understood that various omissions and substitutions of equivalents are contemplated as circumstances may suggest or render expedient.

### DETAILED DESCRIPTION

Approximately one in seven couples has difficulty conceiving. Infertility may be due to a single cause in either partner, or a combination of factors (e.g., genetic factors, diseases, or environmental factors) that may prevent a pregnancy from occurring or continuing. Every woman will become infertile in her lifetime due to menopause. On average, egg quality and number begins to decline precipitously at 35. However, some women experience this decline much earlier in life, while a number of women are fertile well into their 40s. Similarly, while it is normal for women's reproductive lifespans to include periods of natural infertility, associated with menstrual periods or post-partum changes in reproductive endocrinology, for example, some women experience abnormally extended periods of infertility. Such disorders are referred to as infertility-, fecundity-, or fertility-related disorders.

The elucidation of the genetic basis of female fecundity and fertility disorders, particularly the discovery of the key genetic loci underlying these disorders, holds great promise for the identification of novel targets for drug development and therapeutics. More specifically, a better understanding of the crucial molecular pathways underlying human fecundity and fertility guides the next generation of targeted, non-hormonal contraceptives.

The present disclosure is directed to systems and methods for identifying and prioritizing targets for non-hormonal female contraceptive drug development. The non-hormonal female contraceptive drug may be a small molecule, antibody, recombinant protein, or other formulation. They drug may impact any biological processes, including processes involving hormones. In particular, the present disclosure includes a system utilizing genome-wide association studies of all known human genes that have the potential to be pre-fertilization drug targets for female contraceptive development. Genes identified by the system show a genetic or functional association to any phenotype and/or condition related to fertility and/or reproductive health. The drugs may target at least one time point along the spectrum of biological processes that take place during the reproductive cycle, for example, from the formation of the human oocyte to the implantation of the embryo into the uterus. In particular, the system carries out a genome-wide, comprehensive evaluation and screening to uncover genetic factors related to one or more fertility disorders, such as endometriosis, polycystic ovary syndrome, and premature ovarian failure (also known as primary ovarian insufficiency (POI)), for example. The genetic factors may regulate any biological process essential to oocyte development, maturation and fertilization and implantation of the early embryo into the uterus. For example, these processes include, but are not limited to folliculogenesis, oogenesis, oocyte maturation, and ovulation of an egg capable of being fertilized, as well as fertilization, luteinization, endometrial proliferation, and any process by which the endometrium becomes receptive to an embryo. Fertility-related health conditions may involve changes to any of these processes. Hence, the system also identifies genetic factors associated with one or more fertility-related conditions in women for example, those involving ovulatory dysfunction, such as premature ovarian insufficiency (POI), polycystic ovary syndrome, and early menopause, as well as those involving changes to endometrial function, for example endometriosis.

The evaluation and screening processes account for multiple data layers including, but not limited to, temporal and spatial gene expression patterns, pathway-analysis, and protein function. For example, the systems and methods of the invention identify genetic factors or biological molecules and annotate the genetic targets with relevant data from a plurality of data streams. Data streams may include any type of evidence that associates a putative target with a relevant phenotype or condition. Target-phenotype association data streams include, but are not limited to genetic association of target with condition, gene expression data indicating that the target is dysregulated during a pathological state, animal models where the target is modified, and pathway or systems biological frameworks describing the target in disease or normal states. Data streams may also include any type of evidence that helps predict the druggability/tractability of a target and the potential side effect profile if a target is drugged. Target profile data streams include, but are not limited to RNA/protein tissue specificity, and interpreted data from UniProt, HPA, PDBe, DrugEBllity, ChEMBL, Pfam, InterPro, Complex Portal, DrugBank, Gene Ontology, and BioModels.

The systems and methods of the invention rank the genetic targets based on the quantification and strength of evidence relating the target to a relevant biological process or condition. Each independent target-phenotype data element within a given data stream may be assigned a score between 0-1 indicating the strength of the association. Each data stream may be assigned a score between 0-1 that is a result of a harmonic sum function including all data elements within a data stream. An overall association score may also be assigned. For example, the overall association score may be based on a harmonic sum function of all target-phenotype data stream scores. If requirements for targets change, the systems and methods of the invention modify the weighting used to rank the targets to attain a desired output.

Upon identifying genetic factors, specifically key genetic loci underlying one or more fertility disorders, the system is configured to correlate the identified genetic factors with known metabolic pathways and signaling networks, particularly those pathways and networks likely to contain one or more druggable targets (i.e., a biological target, such as a protein, that is known to or is predicted to bind with high affinity to a drug). The system further identifies, from a database of known drug/gene interactions, one or more therapeutic candidates from existing pharmacopeia. The potential targets can then be validated via pre-clinical laboratory partners and/or in silico informatics and analysis techniques.

Accordingly, a pathological basis of infertility is identified (i.e., one or more targets, including genetic factors and/or a pathway known to function in female reproductive biology, thought to be alerted in the context of any female ovulatory phenotype or trait, or thought to be altered in a female fertility disorder) and used as a basis for developing a non-hormonal contraceptive drug in a female who otherwise has normal fertility (i.e., no known fertility disorders). More specifically, based on the identified and prioritized targets, a non-hormonal contraceptive drug can be developed and designed in so as to modulate an otherwise normal functioning signal pathway associated with a menstrual cycle, for example, to thereby alter the cycle and ultimately act as a contraceptive.

Accordingly, the systems and methods of the present disclosure allow for the development of new contraceptive drugs (i.e., non-hormonal contraceptives) with minimal side effects and a more precise mechanism of action, as compared with current hormone-based contraceptives. Furthermore, the present invention has the potential to have a broader impact on women's health beyond contraception, as contraceptives are often used for non-contraceptive purposes in women with reproductive conditions such as endometriosis and polycystic ovary syndrome.

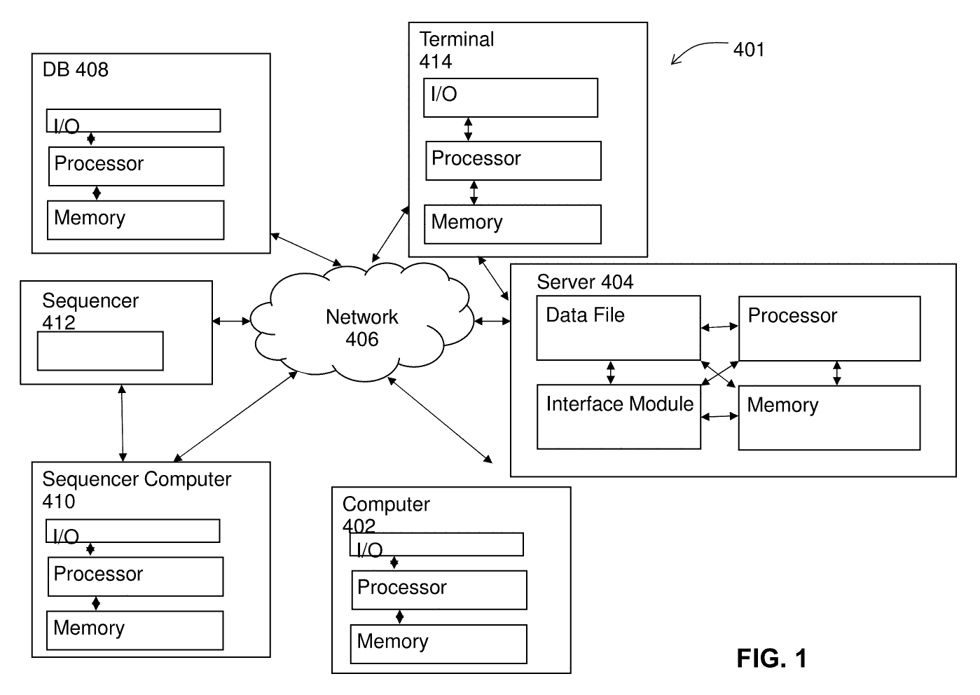

FIG. 1 illustrates a computer system 401 useful for implementing methodologies described herein. A system of the invention may include any one or any number of the components shown in FIG. 1. Generally, a system 401 may include a computer 402 and a server computer 404 capable of communication with one another over network 406. Additionally, data may optionally be obtained from a database 408 (e.g., local or remote). In some embodiments, systems include an instrument 412 for obtaining sequencing data, which may be coupled to a sequencer computer 410 for initial processing of sequence reads.

In some embodiments, methods are performed by parallel processing and server 404 includes a plurality of processors with a parallel architecture, i.e., a distributed network of processors and storage capable of collecting, filtering, processing, analyzing, ranking genetic data obtained through methods of the invention. The system may include a plurality of processors configured to, for example, 1) collect genetic data from different modalities: a) one or more infertility databases 408 (e.g. infertility databases, including private and public fertility-related data), b) from one or more sequencers 412 or sequencing computers 410, c) from mouse modeling, etc; 2) identify a gene or pathway, for example, thought to be altered in a female fertility disorder; 3) expose the gene or pathway to a candidate drug; and 4) select, as a clinical candidate for a non-hormonal female contraceptive, a candidate non-hormonal female contraceptive drug that modulates said gene or pathway.

By leveraging genetic data sets obtained across different sources, applying layers of analyses (i.e., filtering, clustering, etc.) to genetic data, and quantifying/qualifying statistical significance of that genetic data, systems of the invention are able to better screen for a non-hormonal female contraceptive therapeutic. For example, methods of the invention utilize data sets from different modalities. The data sets range include data obtained from infertility databases (e.g., public and private), sequencing data (e.g., whole genome sequencing from one or more biological samples), drug data (e.g., public and private), and genetic data obtained from mouse modeling, etc. Several layers of analysis are then applied to the genetic data by leveraging the Reproductive Atlas platform on the system 401 to perform a genome-wide analysis of all known human genes that have the potential to be pre-fertilization drug targets for female contraceptive development. This genome-wide, comprehensive evaluation and screening of potential targets will include multiple steps that take advantage of multiple data layers including temporal and spatial gene expression patterns, pathway-analysis, and protein function, as will be described in greater detail herein.

While other hybrid configurations are possible, the main memory in a parallel computer is typically either shared between all processing elements in a single address space, or distributed, i.e., each processing element has its own local address space. (Distributed memory refers to the fact that the memory is logically distributed, but often implies that it is physically distributed as well.) Distributed shared memory and memory virtualization combine the two approaches, where the processing element has its own local memory and access to the memory on non-local processors. Accesses to local memory are typically faster than accesses to non-local memory.

Computer architectures in which each element of main memory can be accessed with equal latency and bandwidth are known as Uniform Memory Access (UMA) systems. Typically, that can be achieved only by a shared memory system, in which the memory is not physically distributed. A system that does not have this property is known as a Non-Uniform Memory Access (NUMA) architecture. Distributed memory systems have non-uniform memory access.

Processor-processor and processor-memory communication can be implemented in hardware in several ways, including via shared (either multiported or multiplexed) memory, a crossbar switch, a shared bus or an interconnect network of a myriad of topologies including star, ring, tree, hypercube, fat hypercube (a hypercube with more than one processor at a node), or n-dimensional mesh.

Parallel computers based on interconnected networks must incorporate routing to enable the passing of messages between nodes that are not directly connected. The medium used for communication between the processors is likely to be hierarchical in large multiprocessor machines. Such resources are commercially available for purchase for dedicated use, or these resources can be accessed via “the cloud,” e.g., Amazon Cloud Computing.

A computer generally includes a processor coupled to a memory and an input-output (I/O) mechanism via a bus. Memory can include RAM or ROM and preferably includes at least one tangible, non-transitory medium storing instructions executable to cause the system to perform functions described herein. As one skilled in the art would recognize as necessary or best-suited for performance of the methods of the invention, systems of the invention include one or more processors (e.g., a central processing unit (CPU), a graphics processing unit (GPU), etc.), computer-readable storage devices (e.g., main memory, static memory, etc.), or combinations thereof which communicate with each other via a bus.

A processor may be any suitable processor known in the art, such as the processor sold under the trademark XEON E7 by Intel (Santa Clara, Calif.) or the processor sold under the trademark OPTERON 6200 by AMD (Sunnyvale, Calif.).

Input/output devices according to the invention may include a video display unit (e.g., a liquid crystal display (LCD) or a cathode ray tube (CRT) monitor), an alphanumeric input device (e.g., a keyboard), a cursor control device (e.g., a mouse or trackpad), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

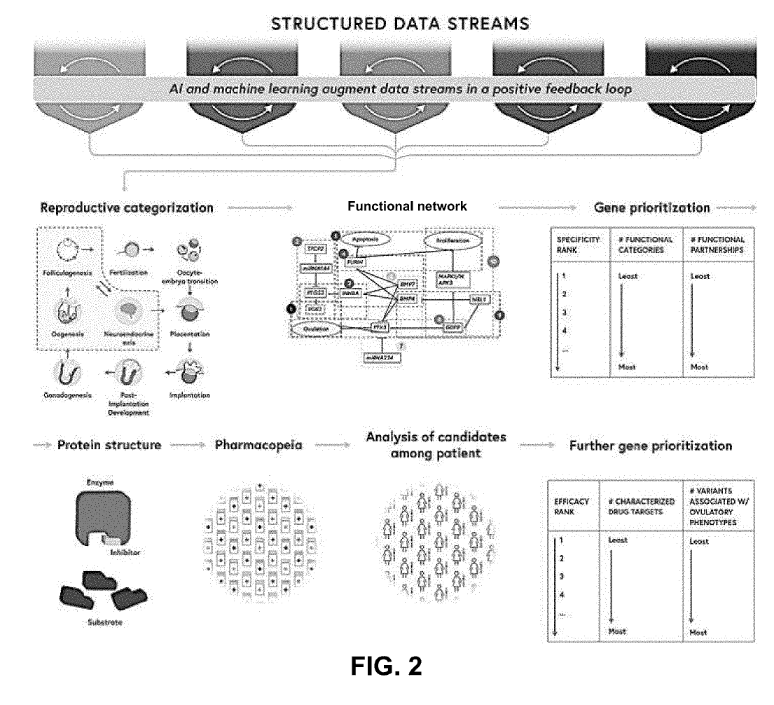

FIG. 2 illustrates various stages of data processing consistent with the systems and methods of the present disclosure, particularly for carrying out the genome-wide, comprehensive evaluation and screening to uncover genetic factors related to one or more fertility disorders, including the determination of specific genes and associated metabolic pathways or signaling networks, and prioritization of such genes based on further processing and refinement.

Genes will be scored and ranked, receiving a higher ranking for having more attributes predicted to positively correlate with contraceptive efficacy. This will include genes known to be disrupted in women experiencing infertility that is refractory to in vitro fertilization treatment. Genes will receive a lower ranking if they are harder to target with small molecules, for example because they are not predicted to have enzymatic activity. They will also receive a greater penalty if they are expressed in more tissues outside of the reproductive system. Genes predicted to function only in post-fertilization events will be excluded. FIG. 2 demonstrates some of these analysis steps and how they flow into iterative scoring and ranking steps.

One major advantage of this approach is that rather than simply outputting a list of genes, we will be tagging each gene with relevant data elements that feed into a flexible ranking algorithm. For example, if during a downstream step, it is determined that genes related to a particular gene network are demonstrating a toxicity profile, a penalty can be imposed for genes with similar features and a new ranked list outputted. If, for example, proteins that are disrupted in women with a specific infertility profile or diagnosis are showing the greatest efficacy in pre-clinical studies, the ranking algorithm can be adjusted to reveal other genes that are similar. The flexible ranking algorithm will be generated that can be tuned to allow differential in silico prioritization based on changing assumptions. Furthermore, a comprehensive, ranked list of potential contraceptive drug targets may be generated through genome-wide analysis. Rather than simply generating a list of genes, the systems and methods of the present disclosure allow for annotating of genes with the relevant data elements needed to rank and output a list of potential contraceptive drug targets. Once the appropriate database has been populated accordingly, an algorithm will be generated to output ranked genes in a flexible way that is responsive to changing assumptions. Success will be tracked by 1) defining the necessary data elements, 2) completing the annotation of genes with these elements, 3) algorithm development and testing, and 4) gene list output.

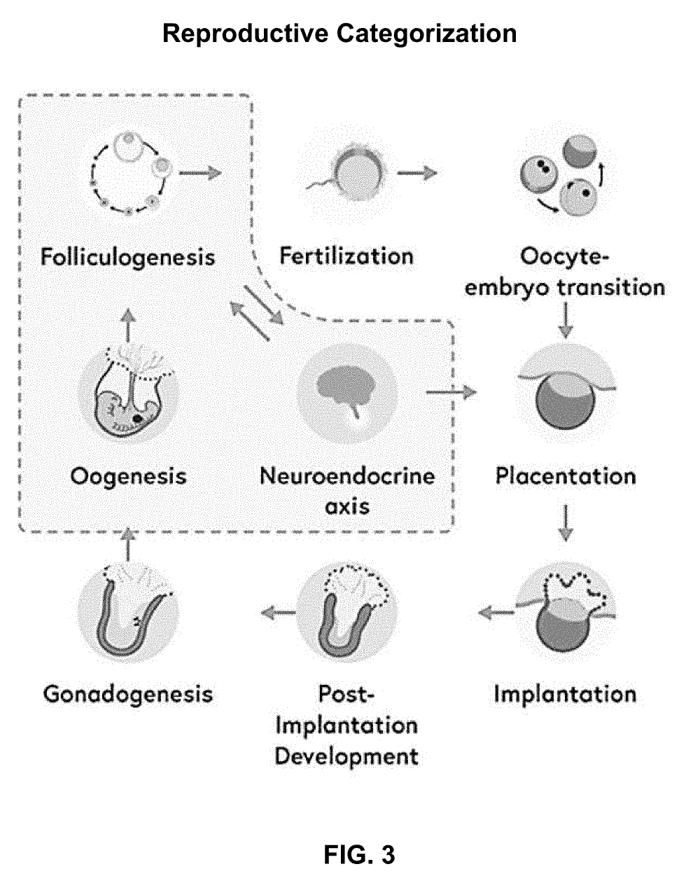

FIG. 3 is an enlarged view of various stages of a reproductive cycle, namely the menstrual cycle, to which various genes and metabolic pathways or signaling networks are related.

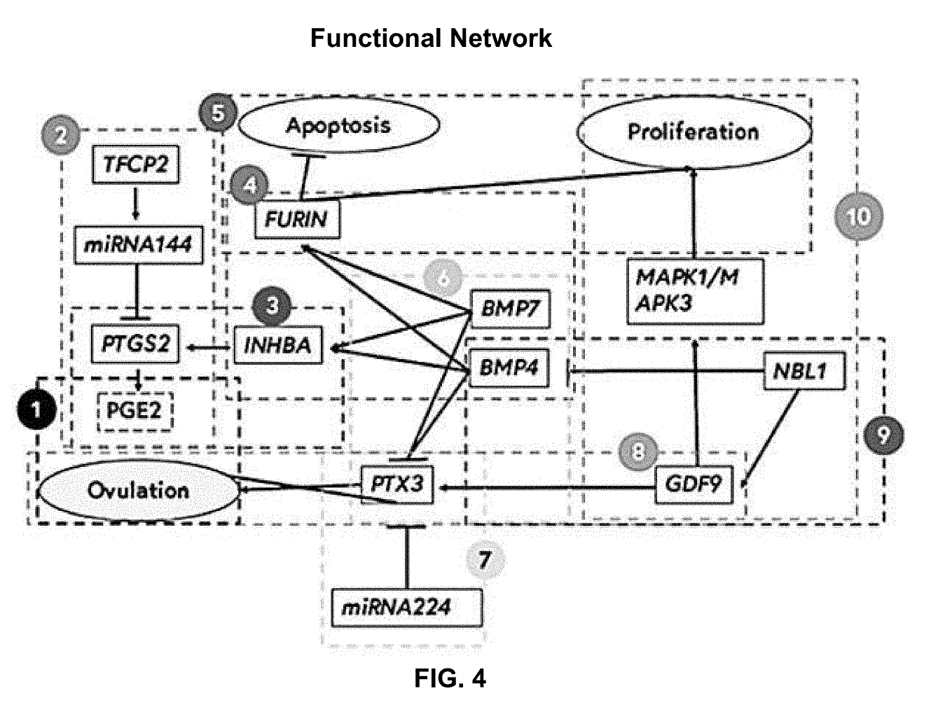

FIG. 4 is an enlarged view of a network of genetic factors, non-genetic factors and biological processes directly or indirectly associated with ovulation identified by systems and methods of the invention. Genetic factors, non-genetic factors and biological processes represent the nodes of the network and the connections among them are represented by edges. Connections are established based on genetic or functional evidence showing that factor or process A may affect factor or process B in any biological context relevant to ovulation.

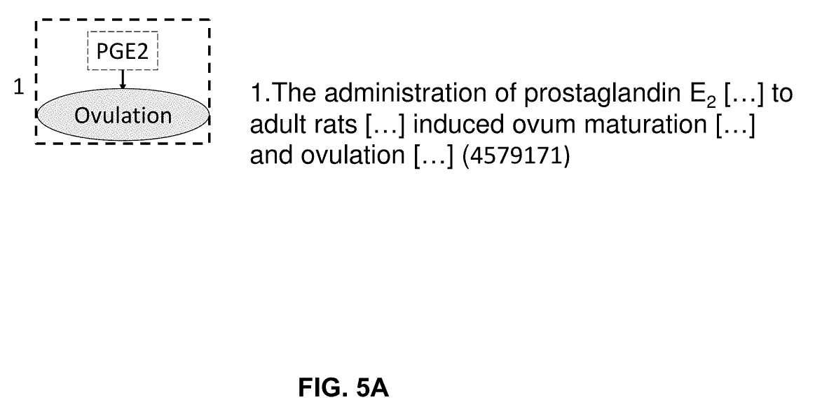

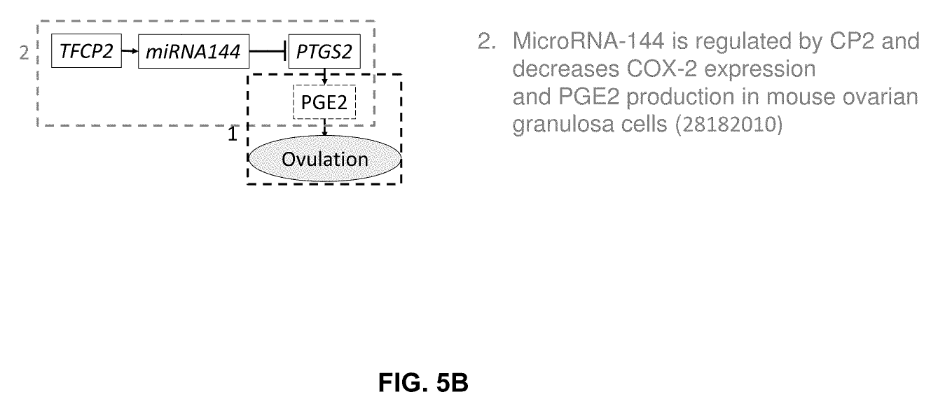

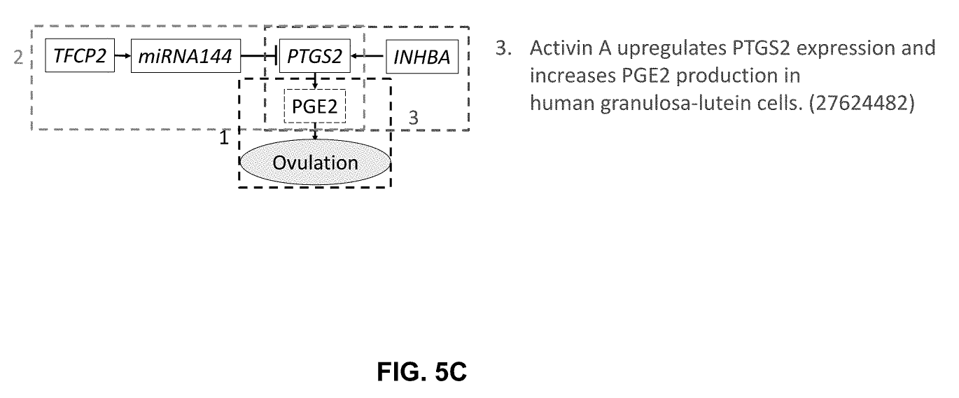

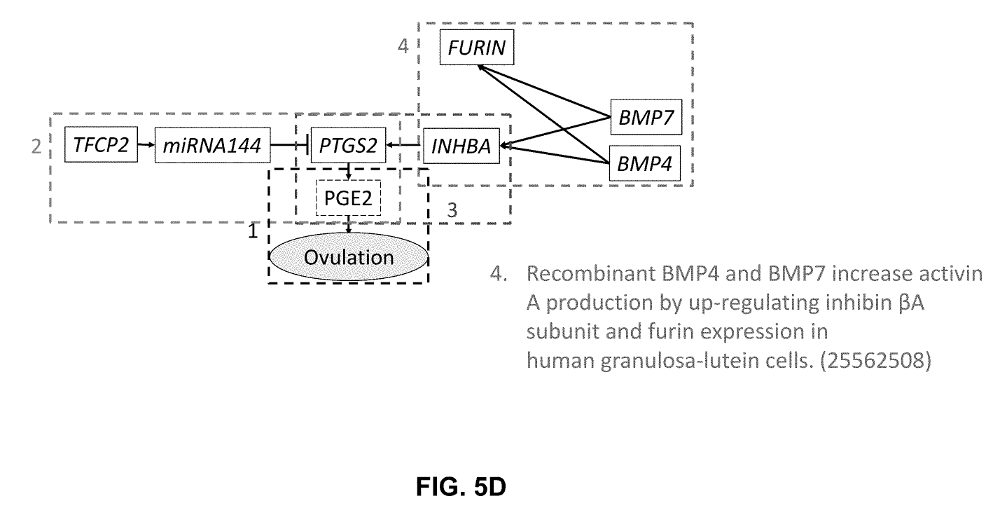

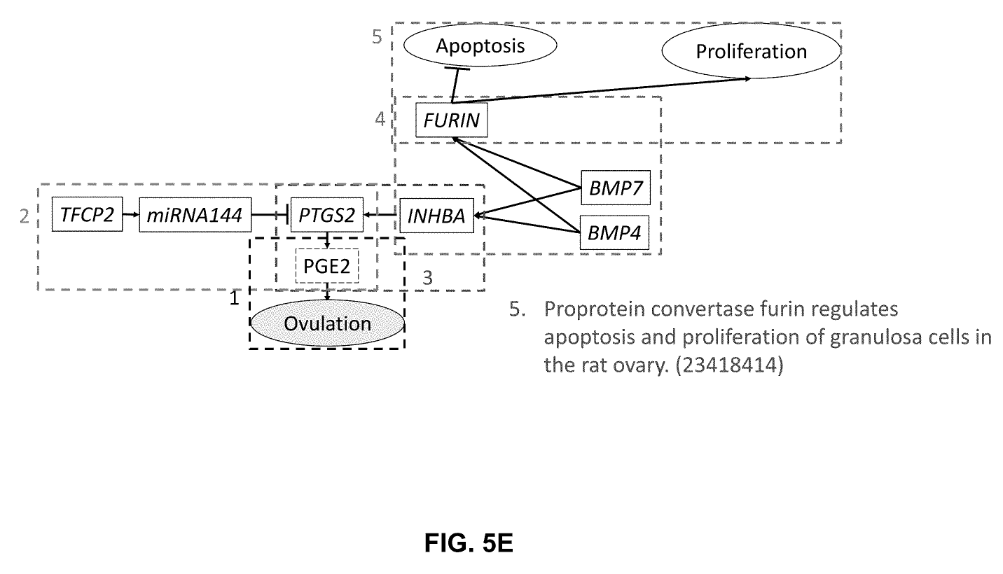

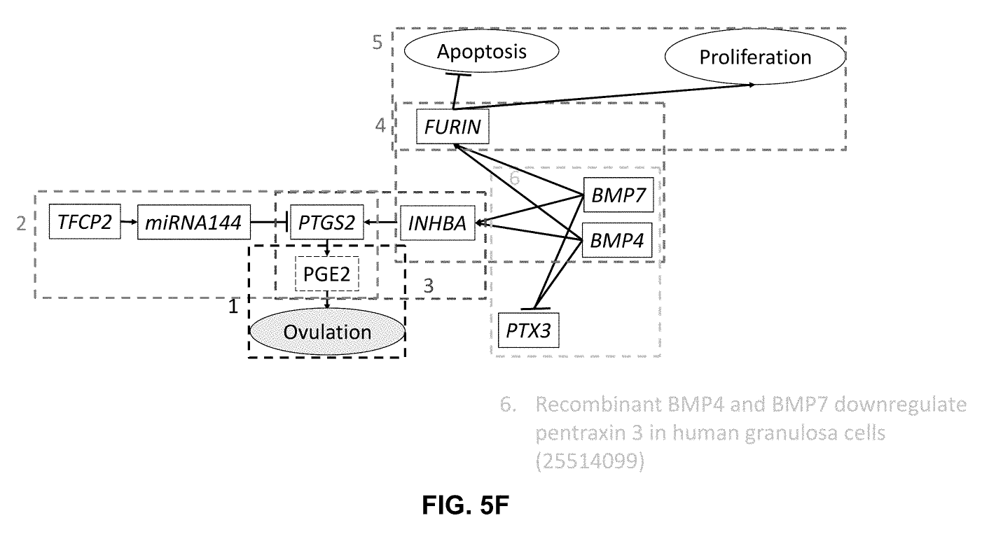

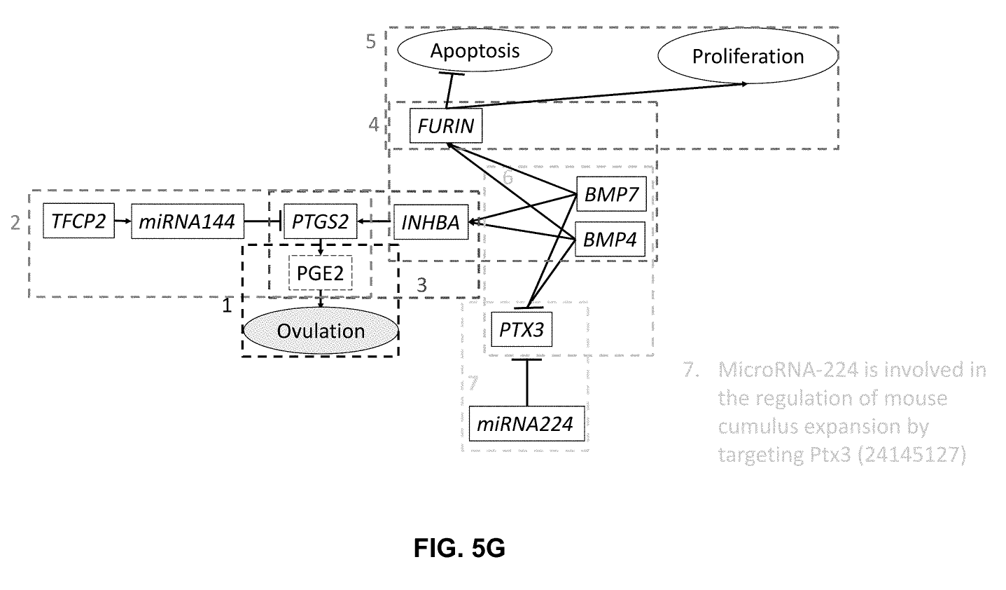

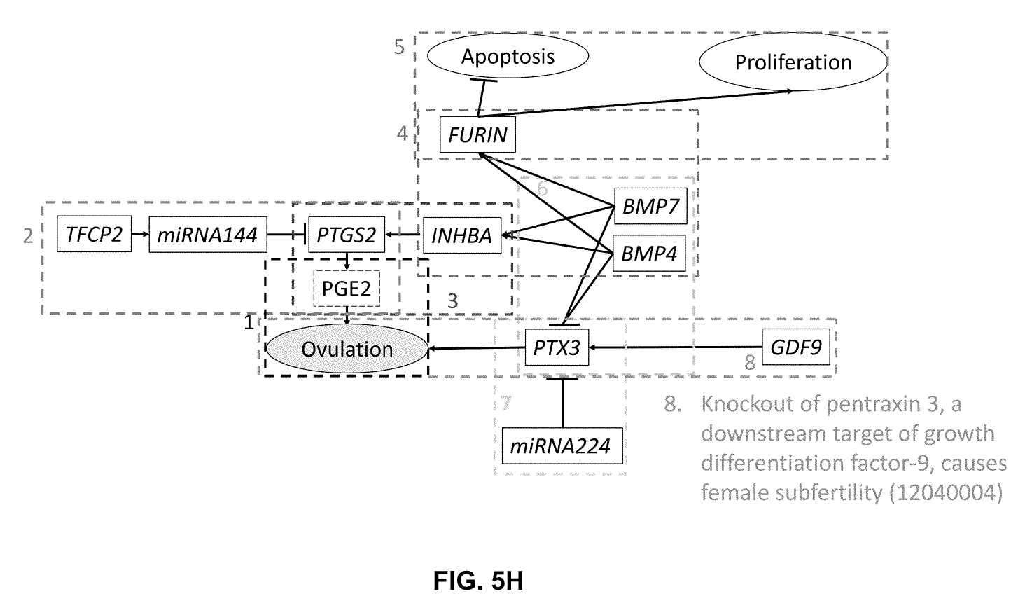

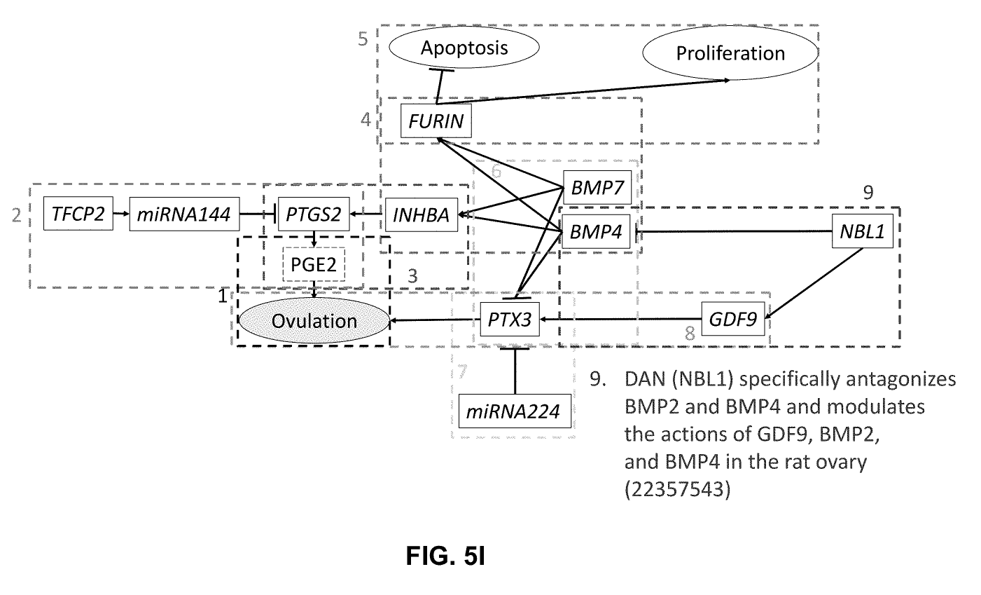

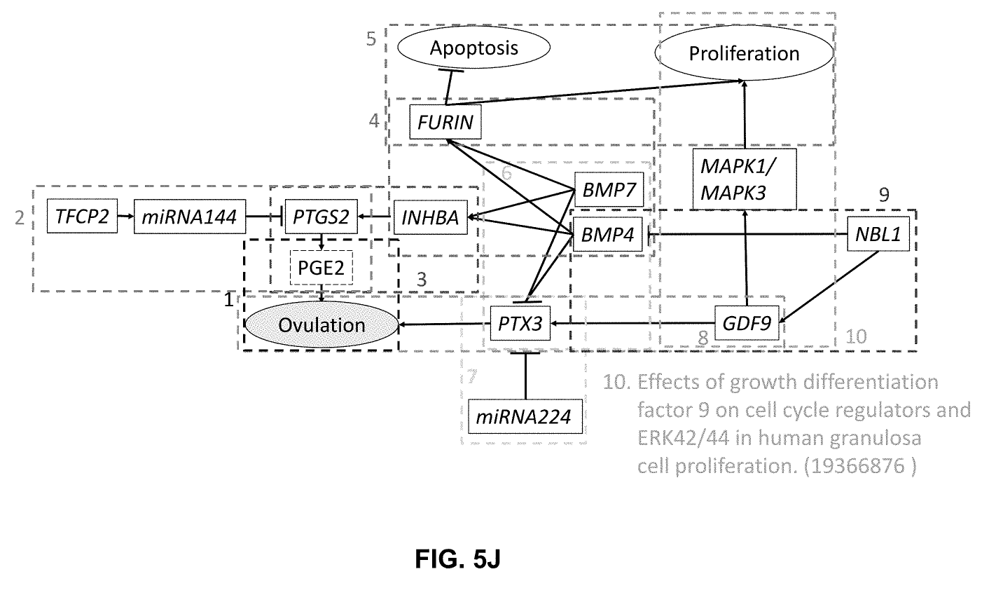

FIGS. 5A-5J are enlarged views of the connections between genetic factors, non-genetic factors and biological processes and a summary of the evidence from which the connections are extrapolated and a summary of the evidence from which the connections are extrapolated is also provided to further illustrate the associated genes.

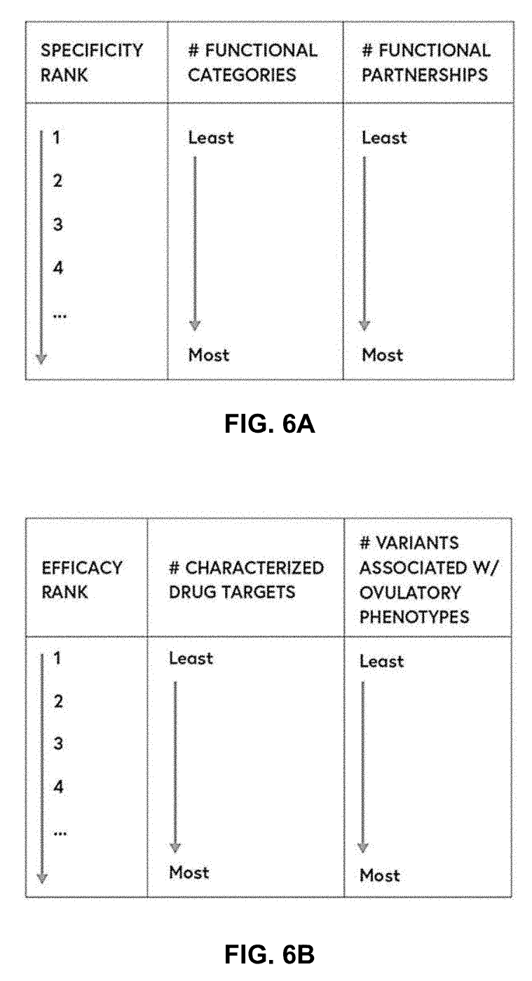

FIGS. 6A and 6B are enlarged views of the gene prioritization tables used in illustrating the ranking of any given identified gene in view of additional processing of related data.

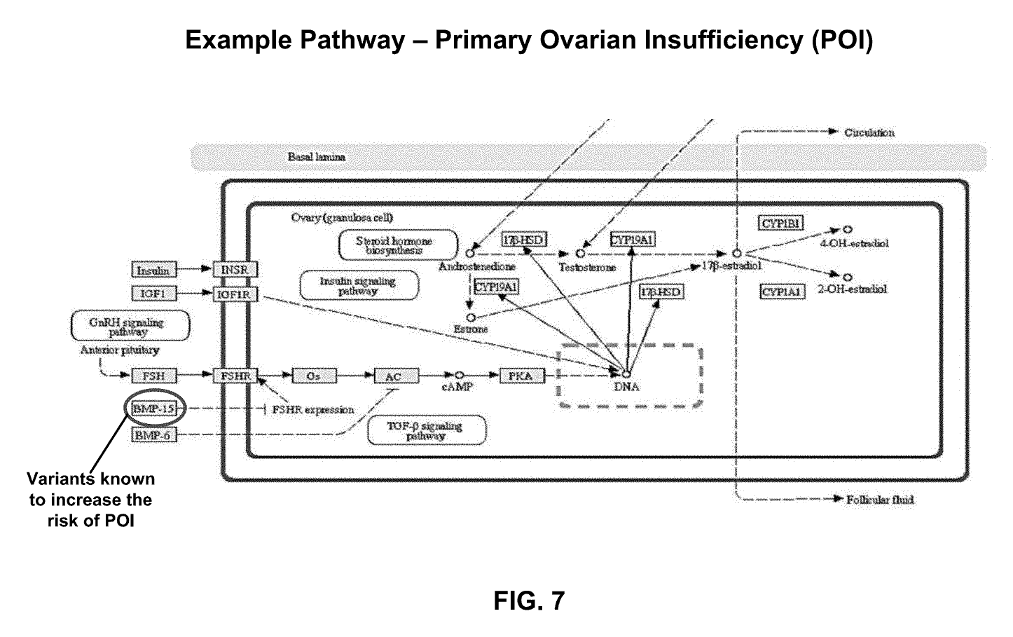

FIG. 7 illustrates an exemplary pathway of the fertility disorder known as primary ovarian insufficiency (POI).

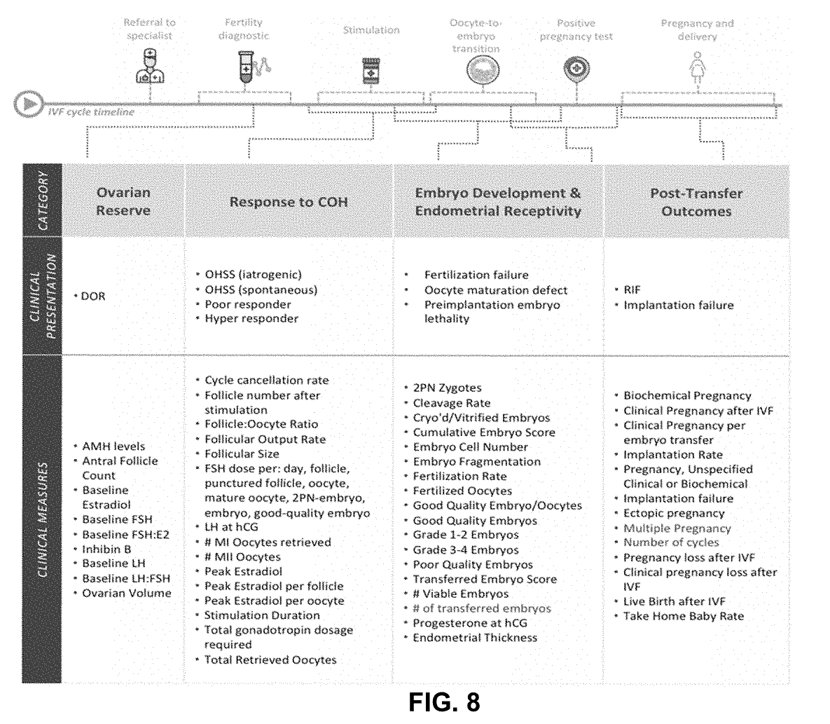

FIG. 8 illustrates an example of a data stream of IVF-related phenotypes and outcomes associated with different genetic alterations for use in identifying candidate targets for non-hormonal contraceptives that affect ovarian or endometrial function.

Accordingly, the systems and methods of the present disclosure allow for the development of new contraceptive drugs (i.e., non-hormonal contraceptives) with minimal side effects and a more precise mechanism of action, as compared with current hormone-based contraceptives. Furthermore, the present invention has the potential to have a broader impact on women's health beyond contraception, as contraceptives are often used for non-contraceptive purposes in women with reproductive conditions such as endometriosis and polycystic ovary syndrome. The elucidation of the genetic basis of female fecundity and fertility disorders, particularly the discovery of the key genetic loci underlying these disorders, holds great promise for the identification of novel targets for drug development and therapeutics. More specifically, a better understanding of the crucial molecular pathways underlying human fecundity and fertility guides the next generation of targeted, non-hormonal contraceptives.

### Example 1—Identifying Gene Targets that May be Candidates for Targeting Ovulatory Function

One way of identifying candidate targets for a non-hormonal contraceptive is to perform a systematic literature review and annotation, in order to identify genes that have published evidence linking them to a human phenotype that mimics the effect of a contraceptive e.g. anovulation or amenorrhea. Genes with the strongest evidence linking them to one of these phenotypes may be prioritized for downstream development. Relevant human phenotypes include those involving ovulatory dysfunction such as, but not limited to, POI, early menopause, PCOS, and diminished ovarian reserve. Relevant human phenotypes may also include those involving altered endometrial function or embryo implantation, or those that involve changes to endometrial receptivity, such as endometriosis.

Candidate genes can also be identified from published studies of women undergoing IVF with controlled ovarian hyperstimulation (COH) that examine the association of genetic alterations with measures of ovarian reserve, response to COH (including poor response and ovarian hyperstimulation syndrome (OHSS)), and outcomes and phenotypes recorded during the course of an IVF cycle, such as ‘follicle:oocyte ratio’, ‘#MII oocytes retrieved’, ‘recurrent implantation failure (RIF)’, or ‘fertilization failure’ (see FIG. 8. for additional examples).

In this example, a systematic review of the literature linking genetic loci to at least of these outcomes or phenotypes, or to POI, PCOS, ovarian aging phenotypes (like early menopause) is provided. Table 1 describes the 607 genes identified with evidence of statistical or functional association with at least one human ovulatory phenotype. The genes listed in Table 1 are candidate targets for a non-hormonal contraceptive designed to affect ovarian function.

TABLE 1
Candidate Target Genes for the Development of Contraceptives Affecting Ovulatory Function

| | | | | | | |
|---|---|---|---|---|---|---|
| PATL2 | C9orf3 | IL11 | DENND1A | CYP2C9 | CCL2 | MT1H |
| TUBB8 | CAPN5 | KCNQ1 | DHCR7 | CYP3A4 | CCL5 | MT1L |
| FSHR | CNR1 | LDLR | DRD3 | CYP3A5 | CD14 | MT1M |
| GDF9 | CYP21A2 | LEP | EPHX1 | DICER1 | CD4 | MT1X |
| LHCGR | FABP4 | LIN28B | EREG | DMC1 | CD40 | MUC1 |
| ACE | FOS | MSH6 | ETV5 | DMRT1 | CD46 | MZB1 |
| ADIPOQ | GHR | NBN | F13A1 | DNAH6 | CD68 | NANOG |
| AKR1C3 | GNRHR | NLRP11 | FAAH | EIF2B3 | CD9 | NCF2 |
| AR | GSK3B | NRIP1 | FAIM2 | FOXO1 | CDKN1A | NCOA3 |
| CAPN10 | GYS2 | PCSK1 | FAS | FOXO4 | CEBPA | NCOA4 |
| CYP11A1 | HMGA2 | PGR | FBN1 | GPR3 | CEBPD | NCOR1 |
| CYP17A1 | HSD17B6 | SMAD3 | FBN2 | HDX | CRP | NGF |
| CYP19A1 | IDE | SMAD7 | FEM1B | HFM1 | CSF2 | NOD2 |
| CYP1A1 | IGF2 | TDRD3 | FOXC2 | HS6ST1 | CTLA4 | NOS1 |
| DENND1A | IL1A | TGFBR1 | FST | HS6ST2 | CTNNB1 | NOS2 |
| ESR2 | KCNJ11 | TLK1 | FSTL3 | IMMP2L | CX3CL1 | NPY |
| FBN3 | KHDRBS3 | TNFSF11 | GHRL | KIT | CXCL1 | ORM1 |
| FEM1A | LEPR | ACSL6 | GNAQ | KITLG | CXCL2 | PCNA |
| H6PD | LMNA | ACVR2B | GNAS | LHX8 | CXCL3 | PEA15 |
| HSD11B1 | LPP | ASH2L | GNB3 | LIN28A | CXCL8 | PI3 |
| IL1B | MCF2L2 | BBS9 | GNPDA2 | MAP4K4 | DHRS9 | PIGU |
| IL6 | MEP1A | BCKDHB | GREB1 | MEIG1 | DKK1 | PIK3CA |
| INS | MIF | BDNF | GSTM1 | MSH4 | DKK2 | PLXNA2 |
| INSR | MMP1 | CDKN1B | HHEX | NANOS1 | DLX4 | PODXL |
| IRS1 | MT-CO1 | CITED2 | HMGCR | NOG | DNAJB1 | POLR2D |
| IRS2 | MT-CO2 | CLPP | HSD17B3 | NXF2 | DUSP12 | PRDX4 |
| MTHFR | MT-CO3 | COMT | ICAM1 | NXF2B | EBP | PSAT1 |
| MTNR1B | MT-CYB | CPEB1 | IGF2BP2 | NXF3 | EGF | PSIP1 |
| PON1 | MT-ND1 | CXCL12 | IKBKG | NXF5 | EGFR | PTBP1 |
| PPARG | MT-ND2 | DACH2 | IL10 | PLCB1 | EPHB2 | PTBP2 |
| SERPINE1 | MT-ND3 | DBH | IL18 | PLP1 | FADD | PTPRC |
| SHBG | MT-ND4 | DIAPH2 | IL6R | POU3F4 | FADS1 | PTX3 |
| SRD5A1 | MT-ND5 | DROSHA | INSIG2 | POU5F1 | FOSB | RBP4 |
| SRD5A2 | MT-ND6 | EIF2B2 | IRF1 | PRL | GAB1 | RHOD |
| TCF7L2 | MT-RNR1 | EIF2B5 | KCTD15 | PTEN | GADD45A | RPL13A |
| THADA | MT-RNR2 | EIF4ENIF1 | LPIN1 | RB1CC1 | GAL | RPS19 |
| TNF | MT-TC | FANCA | LRP5 | RBBP8 | GH1 | RRBP1 |
| VDR | MT-TD | FNDC4 | MAP3K7 | RET | GLB1 | RUNX2 |
| VEGFA | MT-TE | HSD17B4 | MC4R | SKP2 | GNRH1 | S100A6 |
| YAP1 | MT-TK | KDR | MEF2A | SPANXA1 | GPX3 | S100P |
| FMR1 | MT-TQ | LAMC1 | MTCH2 | SPANXA2 | GRB2 | SDHA |
| ALOX12 | MT-TR | LARS2 | MTR | SPO11 | GRHL3 | SERPINA1 |
| AMHR2 | MTNR1A | MCM9 | NR3C1 | STAG3 | GRIN2A | SET |
| APOE | NEGR1 | MMP2 | PAPSS2 | TNXB | GSTM3 | SFRP4 |
| ASH2L | PAFAH1B1 | MMP3 | PDE8A | TSPYL6 | HAS2 | SGCG |
| BMP15 | POMC | MMP9 | PGC | ZFR2 | HDAC3 | SH3D19 |
| BRCA1 | PPARGC1A | MSH5 | PIK3R1 | AGTR1 | HIST2H3C | SLC2A1 |
| BRCA2 | PPP1R3A | MT-ATP6 | PLIN1 | AGTR2 | HMOX1 | SLC2A6 |
| BRSK1 | RAB5B | NANOS2 | PRKAA2 | CTGF | HOXA10 | SOCS3 |
| CYP1B1 | RETN | NANOS3 | PRKAG3 | CXXC5 | HSD3B1 | SOX2 |
| ESR1 | SERPINA12 | NOS3 | PTH | FOXC1 | HSD3B2 | SPARC |
| FMR1 | SGTA | PCDH11X | RAD54B | GAS1 | HSPB1 | SPRR3 |
| HELQ | SORBS1 | PCMT1 | SEC16B | GBP2 | IGFBP2 | STAT3 |
| HK3 | SREBF1 | PGRMC1 | SH2B1 | GREM1 | IGFBP4 | TAB2 |
| IGF1 | SUOX | POF1B | SLC25A5 | IGF1R | IL17A | TCF3 |
| MCM8 | TGFB1 | PSMC3IP | SLC2A4 | NTSR1 | IL1R1 | TFP12 |
| NLRP11 | BLK | RAN | SLC2A4RG | PGRMC2 | IL7 | TGFB2 |
| POLG | CCDC53 | SALL4 | SLC30A8 | PTGER3 | INHBB | THBS1 |
| PRIM1 | DIAPH3 | SETX | STS | PTGS2 | INHBE | THPO |
| PRRC2A | DMPK | SHOX | SULT2A1 | SOCS2 | ITGAL | TIMP1 |
| RHBDL2 | GPR12 | SOHLH1 | TAS2R13 | STAR | ITGAX | TIMP2 |
| SYCP2L | GRIN2B | SOHLH2 | TMEM18 | VCAN | ITGB2 | TM4SF4 |
| TMEM150B | ITIH2 | SYCE1 | TOX3 | ZMIZ1 | ITGB5 | TMEM37 |
| TNFRSF11A | JARID2 | TGIF2LX | TRIB3 | A1BG | JUN | TMPO |
| UIMC1 | KLRA1P | TLK1 | UCP2 | ADAMTS9 | JUND | TNFAIP3 |
| FIGLA | LRRC61 | TSHB | UGT2B15 | ADCYAP1 | LCN1 | TNFAIP6 |
| FOXE1 | MACROD2 | USP9X | VCAM1 | ADIPOR2 | LCN2 | TNIK |
| FOXL2 | MYADML | WNT4 | WFS1 | ALPK3 | LIF | TOP1 |
| GALT | NPR3 | XPO5 | ZP4 | ANGPTL1 | LOX | TPO |
| INHA | TAF4B | WEE2 | ARHGEF7 | AREG | LPL | TWIST1 |
| NOBOX | TMEM86A | AP1 | ALOX15 | ATF2 | MAK | TWSG1 |
| NR5A1 | TP73 | FOLR | APOE | ATP5F1B | MAP2K1 | TXNIP |
| TGFBR3 | TPRXL | FTO | CFTR | BAX | MAPK1 | WT1 |
| FOXO3 | ABRAXAS1 | TP53 | DAZL | BCL2 | MAPK3 | DLX5 |
| SYCP2L | ANKK1 | ABCA1 | EIF4E | BMP2 | MCM6 | DLX6 |
| ZP2 | ARHGS7 | ABCC8 | F2 | BMP5 | MCM7 | FER1L6 |
| ZP3 | CASP8 | ACVR1 | F8 | BMP6 | MITF | FZR1 |
| FSHB | CER1 | ACVR2A | F9 | BMP7 | MMP14 | KISS1R |
| AMH | COL4A3BP | ADIPOR1 | IL1RN | BMP8A | MMP26 | MSH2 |
| TNFA | EIF2B4 | ADRA2B | TNFRSF11B | BMPR1A | MMRN1 | NLRP5 |
| PADI6 | EXO1 | ADRB3 | TP63 | BMPR1B | MRPL13 | NTRK2 |
| TLE6 | F5 | CASR | AIRE | BMPR2 | MS4A1 | PMM2 |
| ADRB2 | F7 | CDKAL1 | BMP4 | C1QBP | MT1A | SEM1 |
| AGT | FANCI | CDKN2A | C4B | C4A | MT1B | SMARCC1 |
| AKT2 | FNDC4 | CDKN2B | COL4A6 | CAPG | MT1E | XPNPEP2 |
| AMH | HDC | CYP2R1 | CSPG5 | CAV1 | MT1F | |
| AQP8 | IGF2R | CYP3A7 | CYP2C19 | CCK | MT1G | |

Once a list of candidate targets (e.g., those in Table 1) has been identified for a particular phenotype, the targets can be ranked using the strength of evidence relating them to a relevant biological process or condition. Evidence is obtained from the different data streams associated with each target. These can include any type of evidence that associates a putative genetic target with a relevant phenotype or condition. Target-phenotype association data streams include, but are not limited to genetic association of a target with condition, gene expression data indicating that the target is dysregulated during a pathological state, animal models where the target is modified, and pathway- or systems-level biological frameworks describing the target in disease or normal states. Data streams may also include any type of evidence that helps predict the druggability and/or tractability of the target, as well as the potential side-effect profile if the target is drugged. Target profile data streams include, but are not limited to RNA/protein tissue specificity, and interpreted data from UniProt, HPA, PDBe, DrugEBllity, ChEMBL, Pfam, InterPro, Complex Portal, DrugBank, Gene Ontology, and BioModels.

Each independent target-phenotype data element within a given data stream may be assigned a score between 0-1 indicating the strength of the association. Each data stream may also be assigned a score between 0-1 that is a result of a harmonic sum function including all data elements within a data stream. An overall association score may be calculated, for example the score may be calculated using a harmonic sum function to add all target-phenotype data stream scores. If requirements for targets change, the systems and methods of the invention modify the weighting used to rank the targets to attain a desired output.

Methods of the invention include combining component variables to calculate the evidence association score for each target-phenotype combination. These variables include: 1) the relative occurrence of evidence supporting a particular target-disease combination (for example, how often the target is observed to altered in case vs control subjects); 2) the predicted functional consequence or severity of the effect of altering the target (e.g. as observed in animal models of the target, or as indicated by the odds ratio or relative risk of the alteration in the target and the phenotype or trait; 3) the overall confidence in the observations that constitute the target disease evidence, for example as indicated by the p-value of individual observations.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof

What is claimed is:

1. A method for screening for a non-hormonal female contraceptive therapeutic, the method comprising:
identifying a gene or pathway known to function in female reproductive biology;
exposing said gene or pathway to a candidate drug; and
selecting, as a clinical candidate for a non-hormonal female contraceptive, a candidate non-hormonal female contraceptive drug that modulates said gene or pathway.

2. The method of claim 1, wherein said gene or pathway is thought to be altered in the context of any female ovulatory phenotype of trait.

3. The method of claim 2, wherein said ovulatory phenotype or trait is a female fertility disorder is selected from the group consisting of endometriosis, polycystic ovary syndrome, and premature ovarian failure or primary ovarian insufficiency (POI).

4. The method of claim 1, wherein identifying said gene or pathway comprises evaluating and screening at least one of temporal and spatial gene expression patterns, pathway analysis, and protein function.

5. The method of claim 1, further comprising identifying a plurality of genes or pathways known to function in female reproductive biology.

6. The method of claim 5, further comprising prioritizing said identified plurality of genes based on a predictive correlation with contraceptive efficacy.

7. The method of claim 6, wherein prioritizing said identified plurality of genes comprises ranking each of said identified plurality of genes based, at least in part, on attributes of each of said identified plurality of genes considered to be associated with contraceptive efficacy.

8. The method of claim 7, wherein a higher ranking corresponds to a more positive correlation with contraceptive efficacy.

9. The method of claim 7, wherein said attributes comprise genes known to be disrupted in females experiencing infertility that is refractory to in vitro fertilization treatment.

10. The method of claim 1, wherein said identified gene is associated with at least one gene listed in Table 1.

11. The method of claim 1, wherein said identified gene is associated with at least one of ADA, AGT, AKT1, ALDOA, AMBP, AMD1, ANXA5, APC, APOA1, APOE, AR, AREG, ATM, ATR, BAX, BCL2, BCL2L1, BDNF, BMP3, BMP4, BMP6, BMP7, BRCA1, BRCA2, BSG, CASP1, CBS, CCL5, CCND1, CCND2, CD19, CD28, CDKN2A, CGB5, COMT, CP, CRHR1, CSF1, CSF2, CX3CL1, CXCR4, CYP11A1, CYP19A1, CYP1A1, DDIT3, DHFR, DNMT1, DPYD, EGR1, ESR1, ESR2, FANCG, FASLG, FDXR, FGFR1, GALT, GATA4, GCK, GGT1, GNRH1, GRN, GSTA1, HBA2, HMOX1, HSD3B2, HSF1, ICAM1, IGF1, IGF1R, IGFBP3, IGFBP4, IL10, IL13, IL1B, ILS, IL6, IL8, IRF1, ITGAV, KIT, KITLG, LEP, LIF, LIFR, MAPK1, MAPK3, MAPK8, MAPK9, MDK, MDM2, MITF, MLH1, MSH2, MST1, MTHFR, MVP, MX1, MYC, NAT1, NCAM1, NOS3, NR5A1, NTRK1, NTRK2, PARP1, PCNA, PGK1, PGR, PRKCB, PRLR, PTGS1, PTGS2, QDPR, SELL, SLC28A1, STATS, STAT6, S, LT1E1, TBXA2R, TG, TNF, TOP2A, TP53, TPMT, TSHB, TYMS, VDR, VEGFA, XDH genes.

11. The method of claim 1, wherein said selected candidate non-hormonal female contraceptive drug upregulates or downregulates a protein associated with said gene or pathway.
